(19) United States
(12) Patent Application Publication
(10) Pub. No.: US 2025/0282705 A1
(43) Pub. Date: Sep. 11, 2025

(54) PREPARATION OF TERMINALLY UNSATURATED (METH)ACRYLATE CROSSLINKERS

(71) Applicant: Röhm GmbH, Darmstadt (DE)

(72) Inventors: Patrik Hartmann, Buettelborn (DE); Christian Maul, Neustadt a. d. W. (DE); Steffen Krill, Muehltal (DE)

(73) Assignee: Röhm GmbH, Darmstadt (DE)

(21) Appl. No.: 18/861,999

(22) PCT Filed: Apr. 28, 2023
(86) PCT No.: PCT/EP2023/061216

§ 371 Date: Oct. 31, 2024

(30) Foreign Application Priority Data
May 6, 2022 (EP) 22171957.8

(51) Int. Cl. C07C 67/26; C07C 67/03; C07C 67/54; C07C 69/54

(52) U.S. Cl. CPC C07C 67/26; C07C 67/03; C07C 67/54; C07C 69/54

(57) ABSTRACT

A process prepares a di(meth)acrylate diester mixture of at least one first di(meth)acrylate diester and at least one second di(meth)acrylate diester. (Meth)acrylic acid reacts with ethylene oxide or propylene oxide in the presence of a first catalyst to obtain a hydroxyalkyl (meth)acrylate mixture, followed by distilling the hydroxy (meth)acrylate mixture to obtain a first top stream component and a first bottom stream component. The bottom stream reacts with (meth)acrylic acid or C1-C4-alkyl (meth)acrylate in the presence of a second catalyst to obtain a mixture of the di(meth)acrylate diester mixture, the first catalyst and the second catalyst. Distilling of the resulting mixture leads to a second top stream of the di(meth)acrylate diester mixture and a second bottom stream of the first and second catalyst. The di(meth)acrylate diester mixture obtained can be used as a crosslinker in reactive resins and polymerization of alkyl(meth)acrylates.

### FIELD OF THE INVENTION

The present invention relates to a process for preparing a di(meth)acrylate diester mixture containing a first and a second di(meth)acrylate diester, by reacting a hydroxyalkyl (meth)acrylate mixture with (meth)acrylic acid and/or a C₁-C₄-alkyl (meth)acrylate. The invention further relates to a di(meth)acrylate diester mixture obtainable by the process of the invention.

### PRIOR ART

Di(meth)acrylate diesters are known as crosslinkers for polyalkyl (meth)acrylates and are frequently prepared by transesterification of methyl (meth)acrylate with a diol or an epoxide or by esterification of (meth)acrylic anhydride with a diol or an epoxide. The prior art describes various processes for this purpose.

DE 1 147 938 describes the preparation of ethane-1,2-diol mono (meth)acrylate (hydroxyethyl (meth)acrylate) and hydroxypropyl (meth)acrylate proceeding from (meth)acrylic acid and an ethylene oxide or propylene oxide in the presence of an iron (III) chloride catalyst. This does not form di(meth)acrylate diesters.

DE 10 2011 005 003 A1 describes a reactor for conversion of alkylene oxides, especially for preparation of glycol esters of (meth)acrylic acid, for example hydroxyalkyl (meth)acrylates. This forms di(meth)acrylate diesters only to a very small degree, if at all.

U.S. Pat. No. 5,001,102 describes an iron catalyst for preparation of unsaturated hydroxyalkyl esters. Di(meth)acrylate diesters are formed only to a very small degree, if at all.

WO 2020/035561 describes the preparation of a di(meth)acrylate diester by reaction of a (meth)acrylic anhydride with an epoxide in the presence of a catalyst and a chromium-containing cocatalyst.

The processes described in the prior art are to some degree inconvenient and costly. They require high-purity products as starting materials and frequently do not permit complete esterification, such that frequently only hydroxyalkyl (meth)acrylates are formed, not di(meth)acrylate diesters.

### OBJECT

There is therefore a need for a process for preparing di(meth)acrylate diesters that has the disadvantages of the processes described in the prior art only to a reduced degree, if at all. In particular, the process is to be performable in a simple and inexpensive manner.

### ACHIEVEMENT OF OBJECT

This object was achieved by a process for preparing a di(meth)acrylate diester mixture, wherein the di(meth)acrylate diester mixture contains the following components (A) and (B):

(A) at least one first di(meth)acrylate diester of the general formula (I)

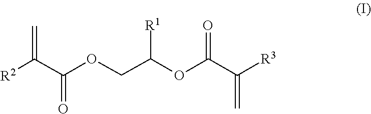

in which
R¹ is H or CH₃,
R² is H or CH₃, and
R³ is H or CH₃;

(B) at least one second di(meth)acrylate diester of the general formula (II)

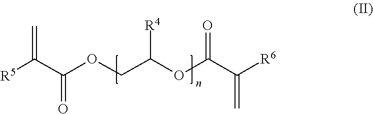

in which
R₄ is H or CH₃,
R₅ is H or CH₃,
R₆ is H or CH₃, and
n is 2 or 3;

wherein the process comprises the following steps:

a) reacting (meth)acrylic acid with at least one epoxide selected from the group consisting of ethylene oxide and propylene oxide in the presence of a first catalyst to obtain a hydroxyalkyl (meth)acrylate mixture, where the hydroxyalkyl (meth)acrylate mixture contains the following components (C) and (D) and the first catalyst:
(C) at least one first hydroxyalkyl (meth)acrylate of the general formula (III)

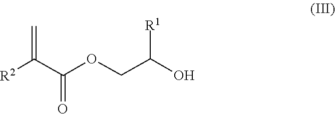

in which
R¹ is H or CH₃, and
R² is H or CH₃;
(D) at least one second hydroxyalkyl (meth)acrylate of the general formula (IV)

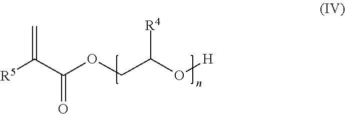

in which
R⁴ is H or CH₃,
R⁵ is H or CH₃, and
n is 2 or 3;

b) distilling the hydroxyalkyl (meth)acrylate mixture obtained in step a) to obtain a first top stream containing component (C), and a first bottom stream containing component (D) and residues of component (C) and the first catalyst,

c) reacting the first bottom stream obtained in step b) with (meth)acrylic acid and/or a C₁-C₄-alkyl (meth)acrylate in the presence of a second catalyst to obtain a mixture containing the di(meth)acrylate diester mixture and the first catalyst and the second catalyst,

d) distilling the mixture obtained in step c) to obtain a second top stream containing the di(meth)acrylate diester mixture, and a second bottom stream containing the first catalyst and the second catalyst.

It has been found that, surprisingly, a first bottom stream formed in the process for preparing a hydroxyalkyl (meth)acrylate can be reacted with (meth)acrylic acid and/or C₁-C₄-alkyl (meth)acrylate to form a di(meth)acrylate diester mixture. This can avoid purely thermal and hence relatively inefficient utilization of the first bottom stream. The di(meth)acrylate diester mixture formed can instead be given a further use, for example as crosslinker. This makes the process of the invention particularly cost-efficient and environmentally benign.

It has also been found that, surprisingly, complex purification of the reactants, especially of hydroxyalkyl (meth)acrylate prior to reaction with (meth)acrylic acid and/or C₁-C₄-alkyl (meth)acrylate can be avoided, which likewise contributes to cost efficiency and economic viability of the process of the invention.

At the same time, the process of the invention permits simple adjustment of the composition of the hydroxyalkyl (meth)acrylate mixture and hence of the di(meth)acrylate diester mixture, such that the di(meth)acrylate diester mixture is reproducible in terms of its composition.

The di(meth)acrylate diester mixture prepared in accordance with the invention can be used as crosslinker, for example in reactive resins and/or in the polymerization of alkyl (meth)acrylates. The polymers obtained have mechanical properties comparable to mechanical properties that are achieved in the case of use of known crosslinkers, such as pure ethylene glycol di(meth)acrylate or pure butane-1,3-or-1,4-diol di(meth)acrylate. At the same time, the preparation of the di(meth)acrylate diester mixtures of the invention is more sustainable.

The process of the invention is elucidated in detail hereinafter.

In step a) of the process of the invention, (meth)acrylic acid is reacted with at least one epoxide selected from the group consisting of ethylene oxide and propylene oxide in the presence of a first catalyst to obtain a hydroxyalkyl (meth)acrylate mixture.

“(Meth)acrylic acid” in the context of the present invention means both acrylic acid and methacrylic acid.

The expression “at least one epoxide” means either exactly one epoxide or a mixture of two or more epoxides. Preference is given to exactly one epoxide.

The at least one epoxide is selected from the group consisting of ethylene oxide and propylene oxide. Ethylene oxide is also referred to as oxirane. Propylene oxide is also referred to as 2-Zethyloxirane.

For example, the molar ratio of (meth)acrylic acid to the at least one epoxide in the reaction in step a) is in the range from 2:1 to 1:2, preferably in the range from 1:0.95 to 1:1.5, and is especially preferably about 1:1.1.

It will be apparent that the molar ratio may vary during the reaction in step a). The stated molar ratio of (meth)acrylic acid to the at least one epoxide in the reaction in step a) is therefore based on the molar ratio before the reaction, i.e. before the (meth)acrylic acid and the at least one epoxide have reacted with one another.

The reaction in step a) is effected in the presence of a first catalyst.

“A first catalyst” in the context of the present invention means either exactly one first catalyst or a mixture of two or more first catalysts. Preference is given to exactly one first catalyst.

It will be clear to the person skilled in the art that, during the reaction in step a), the composition of the first catalyst may vary, for example as a result of transesterification. Therefore, it is possible that the reaction in step a) commences in the presence of exactly one first catalyst, but that the composition of the first catalyst may vary in the course of the reaction, such that a mixture of two or more first catalysts may be formed during the reaction in step a), and then the reaction in step a) takes place in the presence of a mixture of two or more catalysts.

For example, the reaction in step a) is effected in the presence of 0.02% to 2.0% by weight of the first catalyst, preferably in the range from 0.1% to 0.5% by weight of the first catalyst, based in each case on the total weight of (meth)acrylic acid.

Suitable first catalysts are all catalysts known to the person skilled in the art that catalyze the reaction of (meth)acrylic acid with the at least one epoxide.

The first catalyst is preferably a homogeneous catalyst.

A homogeneous catalyst means a catalyst which is fully miscible with and/or can be dissolved in (meth)acrylic acid. For example, the first catalyst may be in solid form under standard conditions and may dissolve in (meth)acrylic acid. It is likewise possible that the first catalyst is in liquid form under standard conditions and is fully miscible with (meth)acrylic acid.

Preference is therefore also given in accordance with the invention to a process in which the first catalyst is a homogeneous catalyst.

The first catalyst is preferably selected from the group consisting of metal salts and organic compounds of metals selected from the group consisting of chromium and iron.

Preference is therefore also given to a process in which the first catalyst is selected from the group consisting of metal salts and organic compounds, each of metals selected from the group consisting of chromium and iron.

Suitable metal salts of metals selected from the group consisting of chromium and iron are, for example, chromium chlorides and/or iron chlorides.

Suitable organic compounds of metals selected from the group consisting of chromium and iron are, for example, chromium(III) acetate, chromium(III) methacrylate, chromium(III) acetylacetonate, chromium(III) formate, chromium(III) acrylate, chromium(III) methacrylate, chromium(III) hexanoate, chromium(III) heptanoate, chromium(III) octanoate, chromium(III) ethylhexanoate, chromium(III) dibutyldithiocarbamate, iron(III) formate, iron(III) acetate, iron(III) acrylate and/or iron(III) methacrylate, particular preference being given to chromium(III) acetate and iron(III) acetate. Also particularly preferred as first catalyst are chromium(III) salts of carboxylic acids.

The reaction in step a) typically takes place at a temperature in the range from 30 to 120° C., preferably in the range from 50 to 100° C.

The pressure in the reaction in step a) is typically in the range from 0 to 6 bar, preferably in the range from 1 bar to 5 bar and more preferably in the range from 2 bar to 3 bar.

The reaction in step a) may take place in reactors for esterifications that are known to the person skilled in the art. Esterifications and reactors for the purpose are known per se and are described, for example, in DE 2011 10005003

The reaction in step a) is also referred to as esterification. In the reaction in step a), the (meth)acrylic acid is esterified with the at least one epoxide. This reaction is known as such to the person skilled in the art.

The reaction forms component (C), at least one first hydroxyalkyl (meth)acrylate of the general formula (III).

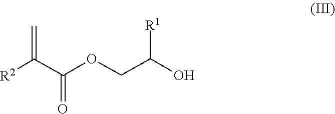

in which

R¹ is H or CH₃, and

R² is H or CH₃.

It will be clear to the person skilled in the art that R¹ is H when the at least one epoxide converted is ethylene oxide. If the at least one epoxide converted is propylene oxide, R¹ is CH₃.

R² is H when the (meth)acrylic acid converted is acrylic acid. If the (meth)acrylic acid converted is methacrylic acid, R² is typically CH₃.

Component (C) is therefore typically selected from the group consisting of hydroxyethyl (meth)acrylate and 2-hydroxypropyl (meth)acrylate.

Hydroxyethyl (meth)acrylate is also referred to as glycol (meth)acrylate or ethylene glycol (meth)acrylate. The term “hydroxyethyl (meth)acrylate” in the context of the present invention means both hydroxyethyl acrylate and hydroxyethyl methacrylate.

2-Hydroxypropyl (meth)acrylate is also referred to as propylene glycol (meth)acrylate or 2-hydroxy-1-propane (meth)acrylate. The term “2-hydroxypropyl (meth)acrylate” in the context of the present invention means both 2-hydroxypropyl acrylate and 2-hydroxypropyl methacrylate.

If the at least one epoxide converted in the reaction in step a) is propylene oxide, there may be formation not only of 2-hydroxy-1-propane (meth)acrylate but additionally also of the corresponding isomer, 1-hydroxy-2-propane (meth)acrylate.

The hydroxyalkyl (meth)acrylate mixture may therefore additionally contain an isomer of the first hydroxyalkyl (meth)acrylate.

The OH group of the first hydroxyalkyl (meth)acrylate of the general formula (III) can react with the at least one epoxide to obtain component (D), at least one second hydroxyalkyl (meth)acrylate of the general formula (IV):

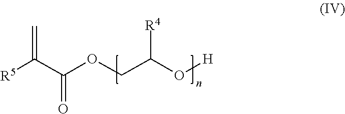

in which

R⁴ is H or CH₃,

R⁵ is H or CH₃, and

n is 2 or 3.

It will be apparent that R⁴ is H when the at least one epoxide converted is ethylene oxide. If the at least one epoxide converted is propylene oxide, R⁴ is CHs.

R⁵ is H when the (meth)acrylic acid converted is acrylic acid. If the (meth)acrylic acid converted is methacrylic acid, R⁵ is typically CH₃.

It will be apparent that, when n in formula (IV) is 2, component (D) typically comprises diethylene glycol mono (meth)acrylate and/or dipropylene glycol mono (meth)acrylate. When n in formula (IV) is 3, component (D) typically comprises triethylene glycol mono (meth)acrylate and/or tripropylene glycol mono (meth)acrylate.

Component (D) is therefore typically selected from the group consisting of diethylene glycol mono (meth)acrylate, triethylene glycol mono (meth)acrylate, dipropylene glycol mono (meth)acrylate and tripropylene glycol mono (meth)acrylate.

The expression “dipropylene glycol mono (meth)acrylate” in the context of the present invention encompasses all isomers of dipropylene glycol mono (meth)acrylate. The expression “dipropylene glycol mono (meth)acrylate” in the context of the present invention thus encompasses the following compounds: 2-(2-hydroxypropan-1-yloxy)-1-propane methacrylate, 2-(1-hydroxypropan-2-yloxy)-1-propane methacrylate, 1-(1-hydroxypropan-2-yloxy)-2-propane methacrylate and 1-(2-hydroxypropan-1-yloxy)-2-propane methacrylate.

The hydroxyalkyl (meth)acrylate mixture obtained in step a) contains, for example, in the range from 90% to 99.99% by weight of component (C) and in the range from 0.01% to 10% by weight of component (D), based in each case on the total weight of the hydroxyalkyl (meth)acrylate mixture.

(Meth)acrylic acid is preferably reacted with exactly one epoxide in step a). Therefore, R¹ in the general formula (III) and R⁴ in the general formula (IV) are preferably the same. Preferably, in addition, R² in the general formula (III) and R⁵ in the general formula (IV) are the same.

Therefore, the hydroxyalkyl (meth)acrylate mixture obtained in step a) preferably either contains hydroxyethyl (meth)acrylate as the first hydroxyalkyl (meth)acrylate and at least one second hydroxyalkyl (meth)acrylate selected from the group consisting of diethylene glycol mono (meth)acrylate and triethylene glycol mono (meth)acrylate or contains propylene glycol (meth)acrylate as the first hydroxyalkyl (meth)acrylate and at least one second hydroxyalkyl (meth)acrylate selected from the group consisting of dipropylene glycol mono (meth)acrylate and tripropylene glycol mono (meth)acrylate.

If the hydroxyalkyl (meth)acrylate mixture contains hydroxyethyl (meth)acrylate as the first hydroxyalkyl (meth)acrylate and at least one second hydroxyalkyl (meth)acrylate selected from the group consisting of diethylene glycol mono (meth)acrylate and triethylene glycol mono (meth)acrylate, the hydroxyalkyl (meth)acrylate mixture in the context of the present invention is also referred to as hydroxyethyl (meth)acrylate mixture.

If the hydroxyalkyl (meth)acrylate mixture contains propylene glycol (meth)acrylate as the first hydroxyalkyl (meth)acrylate and at least one second hydroxyalkyl (meth)acrylate selected from the group consisting of dipropylene glycol mono (meth)acrylate and tripropylene glycol mono (meth)acrylate, the hydroxyalkyl (meth)acrylate mixture in the context of the present invention is also referred to as propylene glycol (meth)acrylate mixture.

The reaction in step a) can be effected in the presence of further components. Such further components are known per se and are, for example, stabilizers.

Suitable stabilizers are especially compounds that inhibit the polymerization of (meth)acrylic acid, the first hydroxyalkyl (meth)acrylate and/or the second hydroxyalkyl (meth)acrylate. The stabilizers are therefore also referred to as polymerization inhibitors.

Suitable stabilizers are, for example, hydroquinone monomethyl ether in conjunction with oxygen, phenol, hydroquinone, nitrophenol and/or butylhydroxytoluene.

The stabilizers used with preference especially include phenol compounds, for example hydroquinones, hydroquinone ethers, such as hydroquinone monomethyl ether, tert-butylhydroquinone, 2,6-di-tert-butylhydroquinone, 2,5-di-tert-butylhydroquinone, 2,4-dimethyl-6-tert-butylphenol or di-tert-butylcatechol; p-phenylenediamines, for example N,N′-diphenyl-p-phenylenediamine, N,N′-di-2-naphthyl-p-phenylenediamine, N,N′-di-p-tolyl-p-phenylenediamine, N-1,3-dimethylbutyl-N′-phenyl-p-phenylenediamine and N-1,4-dimethylpentyl-N′-phenyl-p-phenylenediamine; amines, for example thiodiphenylamine and phenothiazine; copper dialkyldithiocarbamates, for example copper dime-thyldithiocarbamates, copper diethyldithiocarbamates and copper dibutyldithiocarbamates; nitroso compounds, for example nitrosodiphenylamine, isoamyl nitrite, N-nitrosocyclohexylhydroxylamine, N-nitroso-N-phenyl-N-hydroxylamine and salts thereof; and N-oxyl compounds, for example 2,2,4,4-tetramethylazetidine 1-oxyl, 2,2-dimethyl-4,4-dipropylazetidine 1-oxyl, 2,2,5,5-tetra-methylpyrrolidine 1-oxyl, 2,2,5,5-tetramethyl-3-oxopyrrolidine 1-oxyl, 2,2,6,6-tetramethylpiperidine 1-oxyl, 4-hydroxy-2,2,6,6-tetramethylpiperidine 1-oxyl, 6-aza-7,7-dimethylspiro [4,5] decane 6-oxyl, 2,2,6,6-tetramethyl-4-acetoxypiperidine 1-oxyl and 2,2,6,6-tetramethyl-4-benzoyloxypiperidine 1-oxyl; methylene blue, nigrosin base BA, 1,4-benzoquinone, sterically hindered phenols, for example 2,4-dimethyl-6-tert-butylphenol, and/or tocopherol compounds, preferably α-tocopherol.

The stabilizers can be used individually or in the form of mixtures and are generally commercially available. For further details, reference is made to the standard specialist literature, especially to the Römpp-Lexikon Chemie [Römpp's Chemistry Lexicon]; editors: J. Falbe, M. Regitz; Stuttgart, New York; 10th edition (1996); under the heading “Antioxidantien” [Antioxidants] and the references cited there.

For example, the reaction in step a) is effected in the presence of 1 to 5000 ppm by weight of stabilizer, preferably 5 to 1000 ppm by weight, more preferably 10 to 100 ppm by weight, based in each case on the total weight of (meth)acrylic acid.

Preferably, therefore, the hydroxyalkyl (meth)acrylate mixture obtained in step a) contains at least one stabilizer.

In step b), the hydroxyalkyl (meth)acrylate mixture obtained in step a) is distilled to obtain a first top stream and a first bottom stream. The first top stream contains component (C); the first bottom stream contains component (D), residues of component (C) and the first catalyst.

The distillation in step b) can be effected by methods known to the person skilled in the art. For example, the hydroxyalkyl (meth)acrylate mixture obtained in step a) can be transferred to a distillation column and/or a rectification column and distilled therein. This embodiment is preferred in accordance with the invention.

In addition, it is possible that the distillation in step b) is conducted in the same reactor as step a). Then the distillation in step b) is optionally conducted simultaneously with the reaction in step a). The reaction of (meth)acrylic acid with the at least one epoxide in the presence of the first catalyst then takes place simultaneously with the distillation of the hydroxyalkyl (meth)acrylate mixture obtained.

The distillation in step b) can be effected, for example, at a temperature in the range from 70 to 140° C.

The distillation in step b) can be effected, for example, at a pressure in the range from 0 to 100 mbar.

The distillation in step b) affords a first top stream. The first top stream contains component (C), the first hydroxyalkyl (meth)acrylate of the general formula (III).

In addition, a first bottom stream is obtained.

A “bottom stream” in the context of the present invention is understood to mean not just a bottom product which is withdrawn continuously from the distillation in step b), but also a bottom product which, for example in a batchwise mode of operation, remains in the distillation or reactor as distillation residue and is removed only at a later juncture.

The first bottom stream contains component (D), residues of component (C) and the first catalyst. If the hydroxyalkyl (meth)acrylate mixture additionally contains at least one stabilizer, the first bottom stream typically likewise additionally contains the at least one stabilizer.

The expression “residues of component (C)” in the context of the present invention means, for example, 0.1% to 15% of component (C) based on the amount of component (C) present in the hydroxyalkyl (meth)acrylate mixture.

For example, the first bottom stream contains in the range from 40% to 90% by weight of component (C), preferably in the range from 55% to 75% by weight, based in each case on the total weight of the first bottom stream.

For example, the first bottom stream contains in the range from 10% to 65% by weight of component (D), preferably in the range from 15% to 35% by weight, based in each case on the total weight of the first bottom stream.

For example, the first bottom stream contains in the range from 0.1% to 15% by weight of the first catalyst, preferably in the range from 1.0% to 6.0% by weight, based in each case on the total weight of the first bottom stream.

For example, the first bottom stream contains in the range from 0.1% to 15% by weight of the at least one stabilizer, preferably in the range from 0.5% to 6.0% by weight, based in each case on the total weight of the first bottom stream.

The percentages by weight of component (C), of component (D), the first catalyst and of the at least one stabilizer, if any, preferably add up to 100% by weight.

After step b) and before step c), at least one of the following components can be fed into the first bottom stream:

(E) at least one hydroxyalkyl (meth)acrylate selected from the group consisting of ethylene glycol (meth)acrylate and propylene glycol (meth)acrylate and/or

(F) at least one hydroxyalkyl (meth)acrylate selected from the group consisting of diethylene glycol mono(meth)acrylate, triethylene glycol mono(meth)acrylate, dipropylene glycol mono(meth)acrylate and tripropylene glycol mono(meth)acrylate.

Component (E) that can be added to the first bottom stream may be the same as or different than component (C) present in the first bottom stream.

Component (F) that can be added to the first bottom stream may be the same as or different than component (D) present in the first bottom stream.

The addition of at least one of components (E) and/or (F) after step b) and before step c) is advantageous especially in order to adjust the composition of the first bottom stream. In this way, the composition of the di(meth)acrylate diester mixture obtained in the process of the invention can be adjusted.

In step c), the first bottom stream obtained in step b) is reacted with (meth)acrylic acid and/or a C₁-C₄-alkyl (meth)acrylate in the presence of a second catalyst to obtain a mixture containing the di(meth)acrylate diester mixture and the first catalyst and the second catalyst.

The expression “a C₁-C₄-alkyl (meth)acrylate” in the context of the present invention means either exactly one C₁-C₄-alkyl (meth)acrylate or a mixture of two or more C₁-C₄-alkyl (meth)acrylates. “C₁-C₄-Alkyl (meth)acrylates” mean alkyl esters of (meth)acrylic acid that have 1 to 4 carbons in the alkyl radical. The alkyl radical may be linear or branched.

For example, the C₁-C₄-alkyl (meth)acrylate is selected from the group consisting of methyl (meth)acrylate, ethyl (meth)acrylate, n-propyl (meth)acrylate, isopropyl (meth)acrylate, n-butyl (meth)acrylate, tert-butyl (meth)acrylate and isobutyl (meth)acrylate. More preferably, the C₁-C₄-alkyl (meth)acrylate is methyl (meth)acrylate.

The first bottom stream is reacted in step c) with (meth)acrylic acid and/or a C₁-C₄-alkyl (meth)acrylate. The first bottom stream is preferably reacted in step c) either with (meth)acrylic acid or with a C₁-C₄-alkyl (meth)acrylate.

In a preferred embodiment, the first bottom stream is reacted in step c) with acrylic acid and/or a C₁-C₄-alkyl acrylate when acrylic acid has been reacted with the epoxide in step a). It is likewise preferred that the first bottom stream is reacted in step c) with methacrylic acid and/or a C₁-C₄-alkyl methacrylate when methacrylic acid has been reacted with the epoxide in step a). In this embodiment, R² and R³ in formula (I) are the same. It is then likewise the case that R⁵ and R⁶ in formula (II) are the same. Components (A) and (B) present in the di(meth)acrylate diester mixture are then also referred to as symmetric di(meth)acrylate diesters.

In a further, likewise preferred embodiment, the first bottom stream is reacted in step c) with acrylic acid and/or a C₁-C₄-alkyl acrylate when methacrylic acid has been reacted with the epoxide in step a). It is likewise preferred that the first bottom stream is reacted in step c) with methacrylic acid and/or a C₁-C₄-alkyl methacrylate when acrylic acid has been reacted with the epoxide in step a). In this embodiment, R² and R³ in formula (I) are different than one another. It is then likewise the case that R⁵ and R⁶ in formula (II) are different than one another. Components (A) and (B) present in the di(meth)acrylate diester mixture are then also referred to as asymmetric di(meth)acrylate diesters.

For example, the molar ratio of the first bottom stream to the (meth)acrylic acid and/or the C₁-C₄-alkyl (meth)acrylate in the reaction in step c) is in the range from 1:1 to 1:20.

If, in step c), the first bottom stream is reacted with (meth)acrylic acid, for example, the molar ratio of the first bottom stream to the (meth)acrylic acid in the reaction in step c) is in the range from 1:1 to 1:3, preferably in the range from 1:1 to 1:1.5.

If, in step c), the first bottom stream is reacted with C₁-C₄-alkyl (meth)acrylate, for example, the molar ratio of the first bottom stream to the C₁-C₄-alkyl (meth)acrylate in the reaction in step c) is in the range from 1:1 to 1:20, preferably in the range from 1:2 to 1:10.

It will be apparent that the molar ratio may vary during the reaction in step c). The molar ratio of the first bottom stream to the (meth)acrylic acid and/or the C₁-C₄-alkyl (meth)acrylate thus relates to the molar ratio before the reaction, i.e. before the first bottom stream and the (meth)acrylic acid and/or the C₁-C₄-alkyl (meth)acrylate have reacted with one another.

The reaction in step c) is effected in the presence of a second catalyst.

“A second catalyst” in the context of the present invention means either exactly one second catalyst or a mixture of two or more second catalysts. Preference is given to exactly one second catalyst. It will be clear to the person skilled in the art that, during the reaction in step c), the composition of the second catalyst may vary, for example as a result of transesterification. Therefore, it is possible that the reaction commences in the presence of exactly one second catalyst, but that the composition of the second catalyst may vary in the course of the reaction, such that a mixture of two or more second catalysts may be formed during the reaction in step c), and then the reaction in step c) takes place in the presence of a mixture of two or more catalysts.

For example, the reaction in step c) is effected in the presence of 0.1% to 10% by weight of the second catalyst, preferably in the range from 0.1% to 5% by weight of the second catalyst, based in each case on the total weight of the first bottom stream.

Suitable second catalysts are all catalysts known to the person skilled in the art that catalyze the reaction of the first bottom stream, especially of components (C) and (D) present in the first bottom stream, with (meth)acrylic acid and/or the C₁-C₄-alkyl (meth)acrylate.

The second catalyst is preferably selected from the group consisting of Brønsted acids and metal salts and organic compounds of metals selected from the group consisting of tin, titanium, zirconium, alkali metals and alkaline earth metals.

Preference is therefore also given to a process in which the second catalyst is selected from the group consisting of Brønsted acids and metal salts and organic compounds, each of metals selected from the group consisting of tin, titanium, zirconium, alkali metals and alkaline earth metals.

Suitable Brønsted acids are known as such to the person skilled in the art and are selected, for example, from the group consisting of sulfuric acid (H₂SO₄), hydrochloric acid (HCl), para-toluenesulfonic acid, methanesulfonic acid and acidic ion exchangers.

Suitable metal salts of metals selected from the group consisting of tin, titanium, zirconium, alkali metals and alkaline earth metals are known to the person skilled in the art and are, for example, lithium hydroxide, calcium hydroxide, calcium oxide, lithium amide, lithium chloride, sodium hydroxide and/or potassium hydroxide, preferably alkali metal chlorides and/or alkali metal hydroxides, more preferably mixtures of alkali metal chlorides and/or alkali metal hydroxides with alkaline earth metal oxides.

Suitable organic compounds of metals selected from the group consisting of tin, titanium, zirconium, alkali metals and alkaline earth metals are known to the person skilled in the art and are, for example, dibutyltin oxide, dioctyltin oxide, tetraisopropyl titanate and/or zirconium acetylacetonate, preferably tetraisopropyl titanate and/or zirconium acetonylacetonate.

The reaction in step c) is preferably effected in the presence of a Brønsted acid as second catalyst when the second bottom stream is reacted with (meth)acrylic acid.

The reaction in step c) is preferably effected in the presence of a metal salt and/or an organic compound, each of metals selected from the group consisting of tin, titanium, zirconium, alkali metals and alkaline earth metals, when the second bottom stream is reacted with the C₁-C₄-alkyl (meth)acrylate.

The reaction in step c) typically takes place, for example, at a temperature in the range from 60 to 140° C.

If the first bottom stream is reacted with (meth)acrylic acid in step c), the reaction in step c) preferably takes place at a temperature in the range from 60 to 115° C.

If the first bottom stream is reacted with C₁-C₄-alkyl (meth)acrylate in step c), the reaction in step c) preferably takes place at a temperature in the range from 90 to 130° C.

The pressure in the reaction in step c) is typically in the range from 0 to 1500 mbar, preferably in the range from 500 to 1013 mbar.

The reaction in step c) may take place in reactors for esterifications and/or transesterifications that are known to the person skilled in the art. For example, reactors for esterifications may be equipped with water separators, and reactors for transesterification with a suitable column, in order to remove alcohol formed from the reactor.

The reaction in step c) is also referred to as esterification or transesterification. In particular, the reaction in step c) is referred to as esterification when the first bottom stream is reacted with (meth)acrylic acid. The reaction in step c) is referred to as transesterification when the first bottom stream is reacted with C₁-C₄-alkyl (meth)acrylate.

It will be apparent that, in the reaction in step c), component (D) present in the first bottom stream and the residues of component (C) and any components (E) and (F) present are typically reacted with (meth)acrylic acid and/or C₁-C₄-alkyl (meth)acrylate. The first catalyst present in the first bottom stream and the at least one stabilizer present, if any, are preferably not reacted with the (meth)acrylic acid and/or the C₁-C₄-alkyl (meth)acrylate. However, it is possible that any at least one stabilizer present is partly (meth)acrylated.

The reaction in step c) forms the di(meth)acrylate diester mixture. The di(meth)acrylate diester mixture contains components (A) and (B) and is described in detail further down.

What is typically formed is component (A) when the residues of component (C) present in the bottom stream and any component (E) are reacted with (meth)acrylic acid and/or C₁-C₄-alkyl (meth)acrylate. Component (A) is described in detail further down.

What is typically formed is component (B) when the component (D) present in the bottom stream and any component (F) are reacted with (meth)acrylic acid and/or C₁-C₄-alkyl (meth)acrylate. Component (B) is described in detail further down.

As set out above, the first catalyst is typically not converted in step c). The at least one stabilizer present in the first bottom stream, if any, may be partly (meth)acrylated in step c). What is therefore obtained in step c) is a mixture containing the di(meth)acrylate diester mixture, the first catalyst and the second catalyst.

If the first bottom stream contains the at least one stabilizer, the mixture obtained in step c) typically likewise contains the at least one stabilizer; the at least one stabilizer is optionally partly (meth)acrylated.

If the first bottom stream is reacted with (meth)acrylic acid, the reaction typically forms water as a by-product. If the first bottom stream is reacted with C₁-C₄-alkyl (meth)acrylate, the reaction typically forms a C₁-C₄ alcohol as by-product. The C₁-C₄ alcohol is selected, for example, from the group consisting of methanol, ethanol, n-propanol, isopropanol, n-butanol, tert-butanol and isobutanol. The formation of the by-products in the reaction is known as such to the person skilled in the art.

The reaction in step c) thus typically forms a by-product selected from the group consisting of water and a C₁-C₄ alcohol. The mixture obtained in step c) therefore typically additionally contains a by-product. Preferably, after step c) and before step d) or simultaneously with step c), the mixture is distilled to obtain a third top stream containing the (meth)acrylic acid and/or the C₁-C₄-alkyl (meth)acrylate and the by-product, and a third bottom stream containing the di(meth)acrylate diester mixture and the first catalyst and the second catalyst, in which case the third bottom stream is used as the mixture in step d).

The reaction of the first bottom stream in step c) with C₁-C₄-alkyl(meth)acrylate forms a C₁-C₄ alcohol as by-product. In one embodiment, a C₁-C₄ alcohol is thus formed in step c) and, after step c) and before step d) or during step c), the mixture obtained is distilled to obtain a third top stream containing the C₁-C₄-alkyl (meth)acrylate and C₁-C₄ alcohol, and a third bottom stream containing the di(meth)acrylate diester mixture and the first catalyst and the second catalyst and possibly the at least one stabilizer, where the third bottom stream is used as the mixture in step c). It is optionally possible to separate off excess C₁-C₄-alkyl (meth)acrylate from the third bottom stream before it is used as a mixture in step c).

Preference is therefore also given to a process in which the reaction in step c) forms C₁-C₄ alcohol and, after step c) and before step d) or simultaneously with step c), the mixture obtained is distilled to obtain a third top stream containing the C₁-C₄-alkyl (meth)acrylate and C₁-C₄ alcohol, and a third bottom stream containing the di(meth)acrylate diester mixture and the first catalyst and the second catalyst, where the third bottom stream is used as the mixture in step d).

The C₁-C₄-alkyl (meth)acrylate converted is preferably methyl (meth)acrylate. Therefore, the by-product formed is preferably methanol. It is therefore preferable that methanol is formed in step c) and, after step c) and before step d) or during step c), the mixture obtained is distilled to obtain a third top stream containing the C₁-C₄-alkyl (meth)acrylate, especially methyl (meth)acrylate, and methanol, and a third bottom stream containing the di(meth)acrylate diester mixture and the first catalyst and the second catalyst and possibly the at least one stabilizer, where the third bottom stream is used as the mixture in step d).

Preference is therefore also given to a process in which the reaction in step c) forms methanol and, after step c) and before step d) or simultaneously with step c), the mixture obtained is distilled to obtain a third top stream containing the C₁-C₄-alkyl (meth)acrylate and methanol, and a third bottom stream containing the di(meth)acrylate diester mixture and the first catalyst and the second catalyst, where the third bottom stream is used as the mixture in step d).

The third top stream obtained in the distillation can be recycled, for example, into step c).

Preference is therefore also given to a process in which the third top stream is recycled into step c).

The third top stream can be recycled directly into step c). It is likewise possible to purify the third top stream by methods known to the person skilled in the art before it is recycled into step c). In particular, it is preferable to remove the by-product, especially methanol, as far as possible from the third top stream before the third top stream is recycled into step c).

If the first bottom stream is reacted with (meth)acrylic acid in step c), the reaction is preferably effected additionally in the presence of an entraining agent. In this way, it is possible to better separate off the water formed as by-product by distillation in the third top stream. Suitable entraining agents are known as such to the person skilled in the art and are selected, for example, from the group consisting of n-hexane, n-heptane, cyclohexane, methylcyclohexane, toluene and alkyl (meth)acrylates. Alkyl (meth)acrylates suitable as entraining agents are, for example, methyl acrylate, ethyl acrylate and methyl methacrylate, preferably methyl methacrylate.

After step c) and before step d), the first catalyst and/or the second catalyst may be partly separated from the mixture. This affords a mixture having a low catalyst content. In this embodiment, the mixture having a low catalyst content is then used as the mixture in step d)

Preference is also given to a process in which, after step c) and before step d), the first catalyst and/or the second catalyst is partly separated from the mixture to obtain a mixture having a low catalyst content, in which case the mixture having a low catalyst content is used as the mixture in step d).

The first catalyst and/or the second catalyst can be separated off by methods known to the person skilled in the art, for example by means of filtration.

Partial separation of the first catalyst and/or the second catalyst from the mixture affords the mixture having a low catalyst content. The mixture having a low catalyst content contains residues of the first catalyst and residues of the second catalyst. The mixture having a low catalyst content additionally contains the other components that were present in the mixture.

If the mixture is distilled after step c) and before step d) or simultaneously with step c), the first catalyst and/or the second catalyst may be separated partly from the third bottom stream obtained in the distillation to obtain the mixture having a low catalyst content, in which case the mixture having a low catalyst content is used as the mixture in step d). The partial separation of the first catalyst and/or the second catalyst from the third bottom stream obtained in the distillation is correspondingly subject to the observations and preferences described above for the partial separation of the first catalyst and/or the second catalyst from the mixture. In this embodiment, the mixture having a low catalyst content typically contains the di(meth)acrylate diester mixture, residues of the first catalyst and residues of the second catalyst. The mixture having a low catalyst content may additionally contain the at least one stabilizer.

The expression “residues of the first catalyst” means in the range from 0.001% to 100% of the first catalyst, preferably in the range from 0.001% to 10%, more preferably in the range from 0.001% to 2%, based in each case on the total amount of the first catalyst present in the mixture.

The expression “residues of the second catalyst” means in the range from 0.001% to 100% of the second catalyst, preferably in the range from 0.001% to 10%, more preferably in the range from 0.001% to 2%, based in each case on the total amount of the second catalyst present in the mixture.

The first catalyst that has been partly separated off may be recycled, for example, into step a) of the process of the invention. It is likewise possible that the second catalyst that has been partly separated off is recycled into step c) of the process of the invention.

Preference is therefore also given to a process in which the first catalyst and/or the second catalyst is recycled into step a) and/or into step c).

The first catalyst and/or the second catalyst may be purified by methods known to the person skilled in the art prior to recycling into step a) and/or into step c).

In step d) of the process of the invention, the mixture obtained in step c) is distilled to obtain a second top stream and a second bottom stream. The second top stream contains the di(meth)acrylate diester mixture. The second bottom stream contains the first catalyst and the second catalyst.

The distillation in step d) can be effected by methods known to the person skilled in the art. For example, the mixture obtained in step c) can be transferred to a distillation column and/or a rectification column and distilled therein. In addition, it is possible that the distillation in step d) is conducted in the same reactor as step c).

The distillation in step d) can be effected, for example, at a temperature in the range from 70 to 140° C.

The distillation in step d) can be effected, for example, at a pressure in the range from 0 to 100 mbar.

The distillation in step d) affords a second bottom stream. The second bottom stream contains the first catalyst and the second catalyst. If the mixture contains the at least one stabilizer, the second bottom stream obtained in step d) typically likewise contains the at least one stabilizer. The second bottom stream may additionally contain residues of the di(meth)acrylate diester mixture.

The expression “residues of the di(meth)acrylate diester mixture” means in the range from 0.1% to 20%, preferably in the range from 2% to 10%, of di(meth)acrylate diester mixture, based on the total amount of the di(meth)acrylate diester mixture present in the mixture.

It will be apparent that, when the mixture having a low catalyst content has been used in step d), the second bottom stream will contain the residues of the first catalyst and the residues of the second catalyst

The second bottom stream can be recycled, for example, into step a) and/or c). It is possible to purify the second bottom stream by methods known to the person skilled in the art prior to recycling into step a) and/or into step c). In particular, it is possible, prior to the recycling, to separate the first catalyst present in the second bottom stream from the second catalyst present in the second bottom stream. Methods for this purpose are known to the person skilled in the art.

The distillation in step d) affords a second top stream. The second top stream contains the di(meth)acrylate diester mixture.

The second top stream preferably contains in the range from 80% to 99.99% by weight, preferably in the range from 90% to 98% by weight, of the di(meth)acrylate diester mixture, based on the total weight of the second top stream.

The second top stream preferably consists essentially of the di(meth)acrylate diester mixture.

The second top stream may additionally contain residues of the components present in the mixture.

After step d), the second top stream may be purified by methods known to the person skilled in the art to obtain the di(meth)acrylate diester mixture.

The present invention therefore also provides a di(meth)acrylate diester mixture obtainable by the process of the invention.

The di(meth)acrylate diester mixture contains components (A) and (B).

The di(meth)acrylate diester mixture contains, for example, in the range from 50% to 90% by weight of component (A), preferably in the range from 60% to 80% by weight, based in each case on the total weight of the di(meth)acrylate diester mixture.

The di(meth)acrylate diester mixture contains, for example, in the range from 10% to 50% by weight of component (B), preferably in the range from 20% to 40% by weight, based in each case on the total weight of the di(meth)acrylate diester mixture.

Preference is therefore also given to a process in which the di(meth)acrylate diester mixture contains

50% to 90% by weight of component (A) and

10% to 50% by weight of component (B),

based in each case on the total weight of the di(meth)acrylate diester mixture.

The percentages by weight of components (A) and (B) in the di(meth)acrylate diester mixture typically add up to 100% by weight. It is therefore preferable that the di(meth)acrylate diester mixture consists essentially of components (A) and (B).

Component (A) is at least one first di(meth)acrylate diester of the general formula (I)

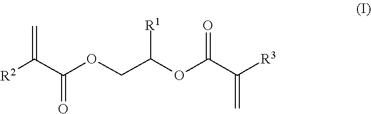

in which

R¹ is H or CH₃,

R² is H or CH₃, and

R³ is H or CH₃.

When, R¹ in formula (1) is H, the first di(meth)acrylate diester is ethylene glycol di(meth)acrylate. Ethylene glycol di(meth)acrylate is also referred to as ethane-1,2-diol di(meth)acrylate.

If R¹ in formula (I) is CH₃, the first di(meth)acrylate diester is propylene glycol di(meth)acrylate. Pro-pylene glycol di(meth)acrylate is also referred to as propane-1,2-diol di(meth)acrylate.

Component (A) preferably comprises ethylene glycol di(meth)acrylate and propylene glycol di(meth)acrylate.

Preference is therefore also given to a process in which component (A) comprises ethylene glycol di(meth)acrylate and propylene glycol di(meth)acrylate.

For example, component (A) comprises in the range from 30% to 80% by weight of ethylene glycol di(meth)acrylate, preferably in the range from 50% to 70% by weight, based in each case on the total weight of component (A)

For example, component (A) comprises in the range from 20% to 70% by weight of propylene glycol di(meth)acrylate, preferably in the range from 30% to 50% by weight, based in each case on the total weight of component (A).

Preference is therefore also given to a process in which component (A) comprises

30% to 80% by weight of ethylene glycol di(meth)acrylate and

20% to 70% by weight of propylene glycol di(meth)acrylate,

based in each case on the total weight of component (A).

Component (B) is at least one second di(meth)acrylate diester of the general formula (II)

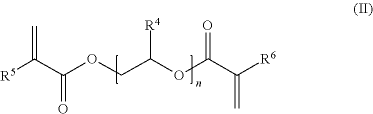

in which

R⁴ is H or CH₃,

R⁵ is H or CH₃,

R⁶ is H or CH₃, and

n is 2 or 3.

When R⁴ in formula (II) is H, the second di(meth)acrylate diester is typically diethylene glycol di(meth)acrylate and/or triethylene glycol di(meth)acrylate. When R⁴ in formula (II) is CH₃, the second di(meth)acrylate diester is typically dipropylene glycol di(meth)acrylate and/or tripropylene glycol di(meth)acrylate. When R⁴ in formula (II) is CH₃, in the context of the present invention, formula (II) and the terms “dipropylene glycol di(meth)acrylate” and “tripropylene glycol di(meth)acrylate” also encompass the corresponding isomers.

It will be apparent that, when n in formula (II) is 2, component (B) typically comprises diethylene glycol di(meth)acrylate and/or dipropylene glycol di(meth)acrylate. When n in formula (II) is 3, component (B) typically comprises triethylene glycol di(meth)acrylate and/or tripropylene glycol di(meth)acrylate.

Component (B) preferably comprises diethylene glycol di(meth)acrylate and dipropylene glycol di(meth)acrylate.

Preference is therefore also given to a process in which component (B) comprises diethylene glycol di(meth)acrylate and dipropylene glycol di(meth)acrylate.

Component (B) comprises, for example, 50% to 90% by weight of diethylene glycol di(meth)acrylate, preferably in the range from 60% to 80% by weight of diethylene glycol di(meth)acrylate, based in each case on the total weight of component (B).

Component (B) comprises, for example, 10% to 50% by weight of dipropylene glycol di(meth)acrylate, preferably in the range from 20% to 40% by weight of dipropylene glycol di(meth)acrylate, based in each case on the total weight of component (B).

Preference is therefore also given to a process in which component (B) comprises

50% to 90% by weight of diethylene glycol di(meth)acrylate and

10% to 50% by weight of dipropylene glycol di(meth)acrylate,

based in each case on the total weight of component (B).

In one embodiment of the present invention, percentages by weight of diethylene glycol di(meth)acrylate and dipropylene glycol di(meth)acrylate in component (B) add up to 100% by weight. It is additionally possible that component (B) comprises further components.

For example, component (B) comprises diethylene glycol di(meth)acrylate, triethylene glycol di(meth)acrylate, dipropylene glycol di(meth)acrylate and tripropylene glycol di(meth)acrylate.

The di(meth)acrylate diester mixture therefore preferably contains

25% to 50% by weight of ethylene glycol di(meth)acrylate,

10% to 35% by weight of diethylene glycol di(meth)acrylate,

0% to 2% by weight of triethylene glycol di(meth)acrylate,

20% to 35% by weight of propylene glycol di(meth)acrylate,

2% to 15% by weight of dipropylene glycol di(meth)acrylate and

0% to 2% by weight of tripropylene glycol di(meth)acrylate,

based in each case on the total weight of the di(meth)acrylate diester mixture.

Steps a) to d) may be conducted batchwise or continuously. The term “batchwise” in the context of the present invention means that the process of the invention is stopped between at least two of steps a) to d). For example, the process of the invention can be stopped between steps b) and c). If steps a) to d) are conducted batchwise, the process of the invention is also referred to as batch process or charge process.

Steps a) to d) are preferably conducted continuously.

The term “continuously” in the context of the present invention means that steps a) to d) are conducted without interruption. Semicontinuous performance is also covered by the term “continuously” in the context of the present invention. For example, it is possible that steps a) and b) are conducted in a batch reactor, but the first bottom stream therefrom is transferred directly into step c). This embodiment is also covered by the term “continuously” in the context of the present invention.

Especially in the case of continuous performance of the process of the invention, all reactants are fed continuously to the various process steps, while all products or product mixtures are simultaneously removed continuously from the process steps and transferred if appropriate into the next process step.

The invention is elucidated in detail hereinafter by examples, without restriction thereto.

### EXAMPLES

### Example 1: Preparation of Hydroxy Ester from Methacrylic Acid and Ethylene Oxide

By the reaction known from the prior art (e.g. DE 2011 10 005 003) for preparation of hydroxy esters from unsaturated acids and epoxides, in four different batches, a reactor was charged with methacrylic acid, and 300 ppm by weight of stabilizer (hydroquinone monomethyl ether, MEHQ) and 0.25% by weight of first catalyst (chromium (III) acetate) were added. At a temperature in the range from 50 to 100° C. and a pressure in the range from 2 to 3 bar, ethylene oxide (molar ratio of methacrylic acid to ethylene oxide 1:1.1) was metered in while monitoring the reaction temperature. Component (C) (hydroxyethyl methacrylate) that was formed in the reaction was essentially separated off as the first top stream in a rectification. The composition of the resultant first bottom stream is reported in table 1.

TABLE 1

| | Batch 1 | Batch 2 | Batch 3 | Batch 4 |
|---|---|---|---|---|
| | [GC | [GC | [GC | [GC |
| First bottom stream | area %] | area %] | area %] | area %] |
| Hydroxyethyl methacrylate content | 61.76 | 67.25 | 48.20 | 41.99 |
| Diethylene glycol | 33.01 | 28.31 | 46.36 | 49.83 |
| monomethacrylate content | | | | |
| Triethylene glycol | 2.20 | 1.72 | 2.97 | 3.20 |
| monomethacrylate content | | | | |
| Other constituents | 3.03 | 2.71 | 2.47 | 4.99 |
| Metal catalyst (Cr) content [%] | 0.43 | 0.40 | 0.60 | 0.61 |

### Example 2: Preparation of Hydroxy Ester from Methacrylic Acid and Propylene Oxide

Analogously to example 1, except using propylene oxide rather than ethylene oxide, the hydroxy ester was prepared from methacrylic acid and propylene oxide. The composition of the resultant first bottom stream is reported in table 2.

TABLE 2

| | Batch 1 | Batch 2 | Batch 3 | Batch 4 |
|---|---|---|---|---|
| | [GC | [GC | [GC | [GC |
| First bottom stream | area %] | area %] | area %] | area %] |
| Hydroxypropyl methacrylate | 80.17 | 64.43 | 66.72 | 71.38 |
| content, sum of isomers | | | | |
| Propylene glycol | 16.57 | 26.53 | 26.72 | 24.28 |
| monomethacrylate content, | | | | |
| sum of isomers | | | | |
| Other constituents | 3.26 | 9.04 | 6.56 | 4.34 |
| Content of the metal | 0.72 | 1.00 | 0.94 | 0.91 |
| catalyst (Cr) [%] | | | | |

### Example 3: Methacrylation of the First Bottom Stream Obtained in Example 1 by Transesterification of Methyl Methacrylate

390.3 g of the first bottom stream from example 1, batch 2 and 900.9 g of methyl methacrylate were heated to boiling at reflux in a 2 liter flask with stirrer and distillation columns. Second catalyst (mixture of 5.47 g of lithium hydroxide and 14.05 g of calcium oxide) was added, and the mixture was again heated to boiling. A third top stream was distilled off at the same time. The third bottom stream obtained (the mixture) was cooled down to room temperature and then filtered. The resultant filtrate was transferred into a second flask and distilled. 466.9 g of the second top stream with the composition specified in table 3 was obtained.

TABLE 3

| Second top stream | [GC area %] |
|---|---|
| Ethylene glycol dimethacrylate content | 69.45 |
| Diethylene glycol dimethacrylate content | 27.26 |
| Triethylene glycol dimethacrylate content | 1.12 |
| Other constituents | 2.17 |

### Example 4: Methacrylation of the First Bottom Stream Obtained in Example 2 by Transesterification of Methyl Methacrylate

432.6 g of the first bottom stream from example 2, batch 2 and 900.9 g of methyl methacrylate were heated to boiling at reflux in a 2 liter flask with stirrer and distillation columns. Second catalyst (mixture of 6.06 g of lithium hydroxide and 15.57 g of calcium oxide) was added, and the mixture was again heated to boiling. A third top stream was distilled off at the same time. The third bottom stream obtained (the mixture) was cooled down to room temperature and then filtered. The resultant filtrate was transferred into a second flask and distilled. 445.5 g of the second top stream with the composition specified in table 4 was obtained.

TABLE 4

| Second top stream | [GC area %] |
|---|---|
| 1,2-Propylene glycol dimethacrylate content | 64.19 |
| Dipropylene glycol dimethacrylate content, sum of isomers | 26.91 |
| Tripropylene glycol dimethacrylate content, sum of isomers | 1.35 |
| Other constituents | 7.55 |

### Example 5: Methacrylation of the First Bottom Stream Obtained in Example 2 by Transesterification of Methyl Methacrylate

432.6 g of the first bottom stream from example 2, batch 1 and 1201.2 g of methyl methacrylate were heated to boiling at reflux in a 2 liter flask with stirrer and distillation columns. Second catalyst (mixture of 1.62 g of lithium amide and 0.54 g of lithium chloride) was added, and the mixture was again heated to boiling. A third top stream was distilled off at the same time. The third bottom stream obtained (the mixture) was cooled down to room temperature and then filtered. The resultant filtrate was transferred into a second flask and distilled. 496.2 g of the second top stream with the composition specified in table 5 was obtained.

TABLE 5

| Second top stream | [GC area %] |
|---|---|
| 1,2-Propylene glycol dimethacrylate content | 86.26 |
| Dipropylene glycol dimethacrylate content, sum of isomers | 11.70 |
| Other constituents | 2.04 |

### Example 6: Methacrylation of a Mixture of the Mixtures Obtained in Example 1 and Example 2 by Transesterification of Methyl Methacrylate

1213.2 g of a mixture of the first bottom streams from example 1, batch 3, and example 2, batch 4, and 2252.3 g of methyl methacrylate were heated to boiling at reflux in a 4 liter flask with stirrer and distillation columns. Second catalyst (mixture of 13.59 g of lithium hydroxide and 34.95 g of calcium oxide) was added, and the mixture was again heated to boiling. A third top stream was distilled off at the same time. The third bottom stream obtained (the mixture) was cooled down to room temperature and then filtered. The resultant filtrate was transferred into a second flask and distilled. 1339 g of the second top stream with the composition specified in table 6 was obtained.

TABLE 6

| Second top stream | [GC area %] |
|---|---|
| Ethylene glycol dimethacrylate content | 32.82 |
| Diethylene glycol dimethacrylate content | 26.93 |
| Triethylene glycol dimethacrylate content | 0.52 |
| 1,2-Propylene glycol dimethacrylate content | 28.11 |
| Dipropylene glycol dimethacrylate content, sum of isomers | 8.69 |
| Tripropylene glycol dimethacrylate content, sum of isomers | 0.13 |
| Other constituents | 2.80 |

1. A process for preparing a di(meth)acrylate diester mixture, the di(meth)acrylate diester mixture comprising:
(A) at least one first di(meth)acrylate diester of the general formula (I) 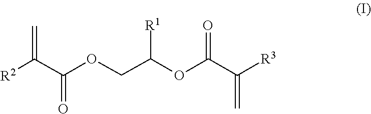
in which
R¹ is H or CH₃,
R² is H or CH₃, and
R³ is H or CH₃;
(B) at least one second di(meth)acrylate diester of the general formula (II) 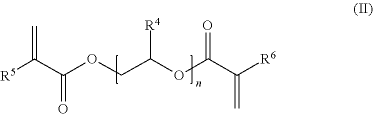
in which
R⁴ is H or CH₃,
R⁵ is H or CH₃,
R⁶ is H or CH₃, and
n is 2 or 3;
wherein the process comprises:
a) reacting (meth)acrylic acid with at least one epoxide selected from the group consisting of ethylene oxide and propylene oxide in the presence of a first catalyst to obtain a hydroxyalkyl (meth)acrylate mixture, the hydroxyalkyl (meth)acrylate mixture comprising:
(C) at least one first hydroxyalkyl (meth)acrylate of the general formula (III) 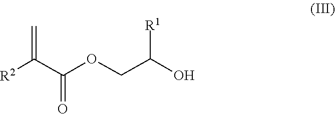
in which
R¹ is H or CH₃, and
R² is H or CH₃;
(D) at least one second hydroxyalkyl (meth)acrylate of the general formula (IV) 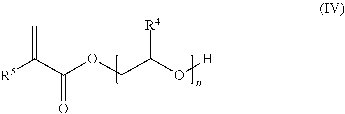
in which
R⁴ is H or CH₃,
R⁵ is H or CH₃, and
n is 2 or 3, and
the first catalyst:
b) distilling the hydroxyalkyl (meth)acrylate mixture obtained in a) to obtain a first top stream containing component (C), and a first bottom stream containing component (D) and residues of component (C) and the first catalyst,
c) reacting the first bottom stream obtained in b) with (meth)acrylic acid and/or a C₁-C₄-alkyl (meth)acrylate in the presence of a second catalyst to obtain a mixture containing the di(meth)acrylate diester mixture and the first catalyst and the second catalyst,
d) distilling the mixture obtained in c) to obtain a second top stream containing the di(meth)acrylate diester mixture, and a second bottom stream containing the first catalyst and the second catalyst.

2. The process as claimed in claim 1, wherein the di(meth)acrylate diester mixture comprises
50% to 90% by weight of component (A) and
10% to 50% by weight of component (B),
based in each case on a total weight of the di(meth)acrylate diester mixture.

3. The process as claimed in claim 1, wherein component (A) comprises ethylene glycol di(meth)acrylate and propylene glycol di(meth)acrylate.

4. The process as claimed in claim 3, wherein component (A) comprises
30% to 80% by weight of ethylene glycol di(meth)acrylate and
20% to 70% by weight of propylene glycol di(meth)acrylate,
based in each case on a total weight of component (A).

5. The process as claimed in claim 1, wherein component (B) comprises diethylene glycol di(meth)acrylate and dipropylene glycol di(meth)acrylate.

6. The process as claimed in claim 5, wherein component (B) comprises
50% to 90% by weight of diethylene glycol di(meth)acrylate and
10% to 50% by weight of dipropylene glycol di(meth)acrylate,
based in each case on a total weight of component (B).

7. The process as claimed in claim 1, wherein the first catalyst is a homogeneous catalyst.

8. The process as claimed in claim 1, wherein the first catalyst is selected from the group consisting of metal salts and organic compounds, each of metals selected from the group consisting of chromium and iron.

9. The process as claimed in claim 1, wherein the second catalyst is selected from the group consisting of Brønsted acids, metal salts and organic compounds, each of metals selected from the group consisting of tin, titanium, zirconium, alkali metals and alkaline earth metals.

10. The process as claimed in claim 1, further comprising:
after c) and before d), partly separating the first catalyst and/or the second catalyst from the mixture to obtain a mixture having a low catalyst content, and
distilling the mixture having a low catalyst content in d).

11. The process as claimed in claim 10, further comprising:
recycling the first catalyst and/or the second catalyst into a) and/or into c).

12. The process as claimed in claim 1, further comprising:
forming a C₁-C₄ alcohol in c) and,
after c) and before d) or simultaneously with c), distilling the mixture obtained to obtain a third top stream containing the C₁-C₄-alkyl (meth)acrylate and C₁-C₄ alcohol, and a third bottom stream containing the di(meth)acrylate diester mixture and the first catalyst and the second catalyst, and
distilling the third bottom stream in d).

13. The process as claimed in claim 1, further comprising:
forming methanol in c) and,
after c) and before d) or simultaneously with c), distilling the mixture obtained to obtain a third top stream containing the C₁-C₄-alkyl (meth)acrylate and methanol, and a third bottom stream containing the di(meth)acrylate diester mixture and the first catalyst and the second catalyst, and
distilling the third bottom stream in d).

14. The process as claimed in claim 12, further comprising:
reccyling the third top stream into c).

15. The di(meth)acrylate diester mixture obtainable by a process as claimed in claim 1.
